(19) United States
(12) Patent Application Publication
(10) Pub. No.: US 2020/0330051 A1
(43) Pub. Date: Oct. 22, 2020

(54) CT Imaging Apparatus with One-Piece Curved X-ray Detector

(71) Applicant: Fei Wang, San Jose, CA (US)

(72) Inventor: Fei Wang, San Jose, CA (US)

(21) Appl. No.: 16/819,132

(22) Filed: Mar. 15, 2020

Related U.S. Application Data
(60) Provisional application No. 62/836,606, filed on Apr. 19, 2019.

(51) Int. Cl. A61B 6/03; A61B 6/02; A61B 6/00

(52) U.S. Cl. CPC A61B 6/035; A61B 6/027; A61B 6/5205; A61B 6/4429; A61B 6/4233

(57) ABSTRACT

A novel in-line X-ray CT imaging apparatus is presented. The X-ray CT imaging apparatus comprises one or a plurality of X-ray sources, one or a plurality of one-piece curved X-ray detectors, a rotational gantry, a translational stage, a computer that is loaded with data acquisition system and CT imaging software. The one-piece curved detector is truly one entity with capability of forming a native curved geometry with pre-determined radius. The detector would have the same or similar electronics to that of a conventional rigid X-ray flat panel that includes a photon conversion phosphorus layer configured to generate light photons in response to radiation. Both X-ray source and detector are mounted on a rotation gantry. X-ray CT 3D image projection data can be acquired while the gantry is rotating and object on the stage is moving translational simultaneously. Using CT software, image reconstruction can be performed at the computer.

### FIELD OF THE INVENTION

The present invention relates to an X-ray Computed Tomography (CT) imaging apparatus that is capable of scanning objects helically; and more particularly, the invention relates to an X-ray CT imaging apparatus using one or a plurality of one-piece curved X-ray detectors to acquire helical projection data.

### BACKGROUND OF THE INVENTION

This application relates generally to system and method of X-ray CT imaging apparatus.

In modern high volume industrial inspection environment, regular 2D X-ray radiography is a common practice for inspection of parts at production line.

However, existing 2D X-ray in-line radiography inspection systems usually can only detect X and Y location of a defect but not depth of defects at Z direction. As a result, some expensive parts have to be discarded to avoid risk whenever a defect is found at X and Y because of no knowledge of Z location of depth. The fact is that various defects can be classified as a critical defect or a non-critical defect depending on the depth under a surface. If somehow user can have defect info at Z location of depth then only parts with a critical defect will be discarded and parts with non-critical defect will be saved. This will save manufacturer a lot of resources.

Computed tomography is a 3D imaging technique that has been widely used in medical, security and industrial field. By analyzing the accumulated 3D data from reconstruction of a matrix, which constitutes a depiction of a density function of the object section being examined, internal defects of an object can be located precisely in 3D location.

Currently a multi-slice or multi-row CT apparatus makes it possible to measure a plural number of projection data simultaneously by dividing detectors into a plural number of rows to obtain a high quality slice images.

In this CT system, its components include X-ray source, fan beam, imaging detector, readout electronics, data acquisition computer, image reconstruction and visualization software etc. X-ray source, fan beam, imaging detector are all in a rotational gantry. And multiple straight imaging detectors mounting together usually generate an approximate curved geometry.

An x-ray source and a detector apparatus are positioned on opposite sides of a portion of an inspection object. The X-ray source generates and directs an x-ray beam towards the object, while the detector apparatus measures the x-ray absorption at a plurality of transmission paths defined by the x-ray beam during the inspection process.

As the x-ray passes through an object near center, the X-ray beam is attenuated. During a scan to acquire x-ray projection data, gantry and whole electrical components mounted inside rotate around a center of rotation axis.

Rotation of gantry and operation of X-ray source is controlled by CT main control system. Gantry motor controller controls the rotation speed and position of gantry.

Read-out electronics and data acquisition system provide digitized data from X-ray detector and send data to a computer. An image reconstructor at computer receives digitized X-ray data and performs high speed image reconstruction. Reconstructed image is then displayed, visualized and analyzed to determine object defect status immediately.

One goal of the present invention is to provide a multi-slice CT imaging apparatus with which it is possible to obtain high quality in-line CT image at an industrial high volume production environment with much lower cost.

Therefore, this kind of CT apparatus can be used to inspect large volume objects at an industrial production line because parts can be transported at certain speed through the gantry during CT scan.

By taking thousands of readings from multiple angles around the object, relatively massive amounts of data are thus accumulated. Multi-slice detector arrays can increase the data rate by scanning a given volume with multiple parallel image slices at the same time.

In order to create better resolution between slices, CT scanners have been developed so that an increased numbers of detectors in the longitudinal axial direction are applied.

In theory, more scan slice would result in more parallel images that can be taken and processed and even faster speed. However, there is a trade-off between number of multiple image slices and quality of each image slice.

When numbers of slices increase, detectors behave more like a larger 2D imager, the X-ray scatter from object becomes increasingly significant. It is especially true when X-ray kV goes beyond about 160 kV in an industrial environment.

A helical scan is a very important in-line non-stop inspection feature in a production line where X-ray inspection data can be taken while object is having a continuous translational motion and gantry is rotating simultaneously.

In this invention, we propose a novel low cost X-ray CT imaging apparatus using one or multiple truly curved X-ray detectors for in-line multi-slice CT application.

As technology advances, it is now possible to make X-ray detector truly curved, bendable or even flexible. A thin-film transistor (TFT) is a special kind of field-effect transistor made by depositing thin films of an active semiconductor layer as well as the dielectric layer and metallic contacts over a supporting substrate. If somehow a flexible or bendable substrate is used then it is possible to make whole detector device also flexible and truly curved.

These kinds of flexible X-ray detectors will be thin, lightweight, conformable and highly rugged devices. It was reported that some of the new detectors can be on plastic material of Polyethylene Naphthalate (PEN) and some can on plastic material of Polyimide. So these bendable substrates can be with organic materials.

In fact, there are already curved monitor and folded cell phone display in the market. In X-ray application, it has been reported that panel bending radius now could be down to below 100 mm; panel substrate thickness could be down to sub 100 μm just as a sheet of paper; They have better electrical performance than conventional a-Si glass-based TFT technology; its manufacturing yield can be higher compared with others; therefore, flexible sensor arrays can have much lower total cost of ownership.

Flexible sensor arrays now have opened a new frontier in X-ray imaging application. Flexible image sensors and sensor arrays enable precision imaging in ways simply not possible with rigid glass arrays before. Sometimes it can reach a location that only flexible film can reach. But the flexible detector is now a digital imaging device, while film is still an analog imager that cannot be used for CT application.

GdOS: Tb (GOS) or GdOS: Pr (GOS) material is standard scintillator for X-ray application. GOS screen can also be slightly curved to attach X-ray detector. Plastic materials have the advantage of being lightweight, robust, more sensitive and lower cost than rigid amorphous silicon on glass-based flat panel detector (FPD). This technology is also scalable to enable area detectors to be very large size. The flexibility of the FPDs makes them suitable for the development of conformable detectors that can go around objects for non-destructive testing (NDT) or security applications. In addition, the ultra-low leakage advantage of OTFT (Organic Thin-Film Transistor) compared to amorphous silicon brings directly sensitivity benefits to X-ray detection.

To increase CT data rate, readout electronics architecture has to be in such a way so that its readout is as parallel as possible. With 5G wireless transmission being available now, it is easier to transfer data from detector to computer than ever before.

In this invention, electrically connecting a multi-slice CT detector module to a CT system becomes even simpler in order to avoid bulky electronics structure at the gantry.

An industrial CT (iCT) system cost would be much lower if this kind of truly curved detector is arranged. A layer of low cost scintillating material, such as GOS or other scintillating material is placed on the image sensor to convert the impinging X-ray energies into visible light which can be detected efficiently by the image sensor array. A protective metal shield is fastened to the substrate to protect the sensitive circuits of the image sensor from X-ray radiation damage. A proper separation of sensitive circuits from the photodiode array on the sensor pixel arrays, coupled with precision registration of the sensor chips on the substrate, allows easy installation of a curved geometry on a gantry.

In prior arts, there are a number of different kinds of detectors associated with a multi-slice CT. Popular approaches are to use 2D discrete photodiode arrays or integrated 2D imager array.

One disadvantage in prior arts is that 2D photodiode detector arrays are usually not integrated and need bulky peripheral electronics mounted at the gantry. Their cost is relatively high and machine assembling is quite complicated and time-consuming.

Another disadvantage of 2D photodiode detector arrays or integrated 2D array in prior arts is that it is difficult to make CT detector with truly curved geometry. Their so-called “curved” geometry is actually formed by multiple small straight sections of flat and straight detector sections. They are not really truly curved on rotating gantry. Geometric distortion will have impact on image quality.

Still another disadvantage in prior arts is that there always exist various gaps during assembly in order to make up a curved shape. Gaps between small detector modules are classified as dead space in CT imaging system. The dead space would not only affect software algorithms but also overall image quality.

Still another disadvantage in prior arts is that when pixel size becomes smaller, level of difficulties goes up significantly and very quickly. Discrete photodiode arrays would need more electronics channels and more part pieces. The overall size goes even more bulky at the gantry. Gaps among modules become very significant even for integrated 2D imager array. At some point, software would not be able to correct artifacts based on the fact that there is too much built-in dead space at detector.

In this current invention, we easily use a truly one-piece curved X-ray detector for in-line multi-slice CT imaging apparatus.

The first advantage is that the cost of CT imaging machine will be much lower. Machine cost comprises cost of parts and cost of labor for assembling. Curved detector is just one piece of parts from massive production and it would be much easier to assemble detector at a CT machine.

The second advantage is that detector is with truly curved geometry. The detector pixels are arranged along longitudinal axial direction and direction that is tangent to a circle, and it is much easier for software to do image reconstruction mathematically with much less geometric distortion.

The third advantage is better scalability and configurability. Its radius can be adjusted to fit various gantry sizes without too much effort. Therefore, machine with different gantry size can be more easily configured and built.

The fourth advantage is that there are no gaps or much less gaps at detector or between detectors. It will be also much easier for software to do image reconstruction. Better image reconstruction will result in better image quality in the end.

The fifth advantage is that numbers of slices usually can also be controlled by active region setting or by collimation. Software can also select the active region at the detector. Readout electronics would be similar to that of regular rigid flat panel at which selection of region of interest (ROI) for optimization is common practice.

The sixth advantage is that effective size of pixel elements can also be adjusted by binning feature just like that in a rigid flat panel detector. For example, 2×2 pixel binning, 4×4 pixel binning is very common. So CT machine has built-in capability of variable pixel size.

The seventh advantage is better reliability. In general, less parts in a machine, more reliable it becomes. It is particularly true for X-ray detector modules and electronics modules inside a CT machine.

The eighth advantage is easier to debug in case of issues. Debugging a problem in a complicated machine is always a headache, sometimes could be a nightmare. With one-piece curved detector in the CT machine, it will be much easier.

The ninth advantage is easier to do machine part replacement in case of issues. Just like that of replacing standard parts at standard PC, the one-piece curved detector could become commodity very quickly once an international industrial standard is established.

The tenth advantage is that design of such one-piece curved detector allows it also be used in other industrial applications for better cost-sharing purpose. Shape of the one-piece curved detector when it is flat is usually rectangle which size is similar to that of film cassette or that of screen cassette of Computed Radiography (CR) at current pipeline welding inspection in oil & gas or energy utility industry. This size similarity arrangement is very desirable for flexibility requirement. This kind of one-piece curved detector can be easily mounted on curved structure at rotation gantry to achieve multi-slice CT functionalities; it can also be mounted at a curved oil or water pipe to do low cost welding inspection. Once applications from different fields can share the same parts then production volume of the part can go up quickly, therefore, cost of one-piece curved detectors can go down even lower for X-ray CT imaging apparatus application.

Use of multiple X-ray source configurations is also an option. Multiple X-ray sources would also need multiple one-piece curved detectors.

### BRIEF DESCRIPTION OF THE DRAWINGS

FIG. 1 shows a CT imaging apparatus with one-piece curved X-ray detector.

FIG. 2 shows one set of one-piece curved X-ray detector.

FIG. 3 shows configuration of two sets of one-piece curved X-ray detectors that are positioned head-to-head.

FIG. 4 shows cross section of a CT imaging apparatus with one-piece curved X-ray detector.

FIG. 5 shows cross section of a CT imaging apparatus with multiple X-ray sources and multiple one-piece curved X-ray detectors.

### SUMMARY OF THE INVENTION

The present invention is directed to a system and method for providing lower cost and easier assembly in X-ray in-line CT imaging apparatus.

An X-ray CT imaging apparatus has one or a plurality of sets of one-piece curved detectors arranged in a plural number of rows in a longitudinal axial direction, and moving object is to be also examined for movement in the longitudinal axial direction.

X-ray transmits through the object while X-ray source and the detector on the gantry are rotating. The detector generates signals in response to X-ray radiation beam. So a plural number of helical data projections are acquired.

The one-piece curved detector would have the similar electronics to that of a conventional rigid X-ray flat panel. It includes a photon conversion phosphorus layer configured to generate light photons in response to a radiation.

Both X-ray source and one-piece curved detector are mounted on a rotation gantry. X-ray 3D CT imaging can be achieved while the gantry is rotating and object is moving translational simultaneously.

Various other features and advantages of the present invention will be made apparent from the following detailed description and the drawings.

### DETAILED DESCRIPTION OF THE INVENTION

The present invention is directed to a system and method for providing lower cost and easier assembly in X-ray in-line multi-slice CT imaging apparatus.

The X-ray CT imaging apparatus comprises an X-ray source 1, a one-piece curved X-ray detector 3, a rotational gantry 6, a translational stage 7 on which an object 8 is placed, a computer 9 loaded with data acquisition system and CT imaging software.

In this invention, one or a plurality of one-piece curved X-ray detectors 3 can be used to achieve low cost multi-slice CT imaging functions with much precise geometry.

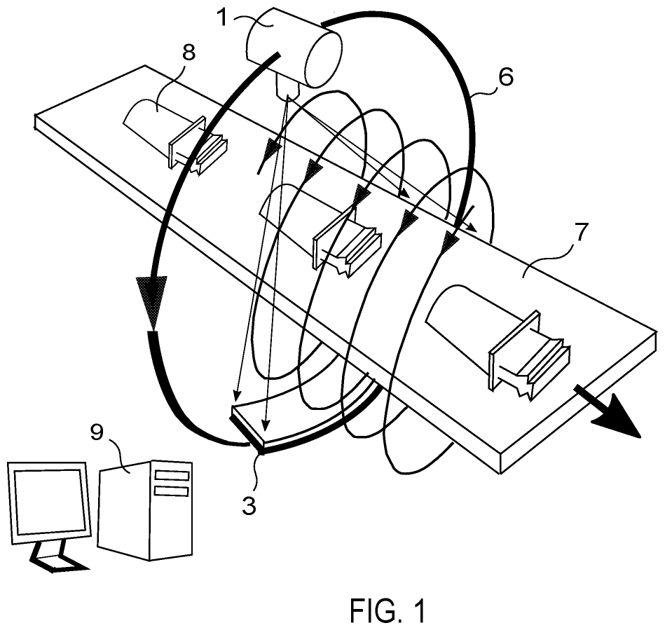

Referring to FIG. 1, it shows a CT imaging apparatus using one X-ray source 1 and one set of one-piece curved X-ray detector 3. The X-ray source 1 and one-piece curved detector 3 can be easily mounted on a rotational gantry 6 with predetermined radius. Therefore, a multi-slice CT imaging apparatus can be easily built.

To construct, firstly, positioning an X-ray source 1 on one side of rotational gantry 6; secondly, positioning one-piece curved detector 3 on another side of rotational gantry 6; thirdly, positioning an object 8 on a translational stage 7; finally, connecting imaging data acquisition hardware and starting data acquisition software. There is a computer 9 that is pre-loaded with imaging data acquisition hardware and software system. The data acquisition can be with either wired connection or wireless connection.

To operate, firstly, rotating the rotational gantry 6 using a motion control system; secondly, moving said translational stage 7 along longitudinal axial direction of said gantry using a motion control system; thirdly, acquiring X-ray helical projection data of the object using the computer 9 while the rotational gantry 6 is rotating, the translational stage 7 is in motion, the X-ray source 1 is emitting X-ray beam 2 and the one-piece curved detector 3 is active; then, performing correction processes to X-ray helical projection data; and finally performing tomography image reconstruction from corrected X-ray helical projection data.

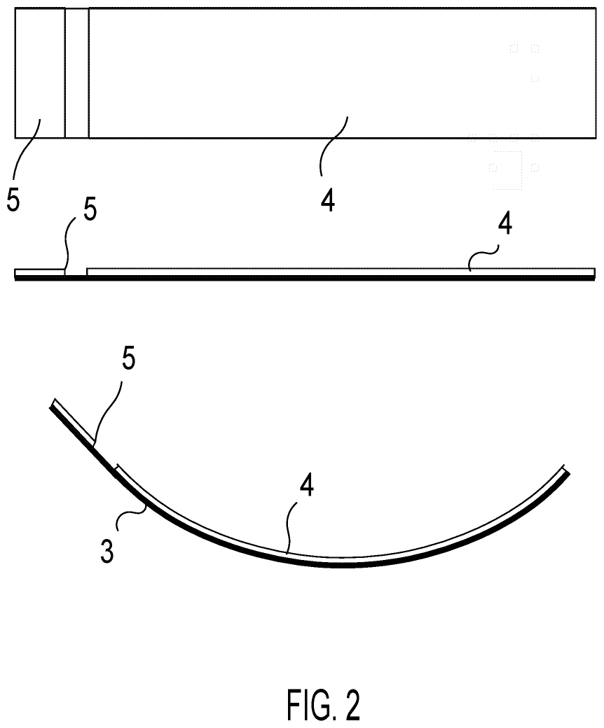

FIG. 2 shows one set of one-piece curved X-ray detector 3. The detector is manufactured into flat shape together with a layer of X-ray scintillator 4 and electronics board 5 mounted originally. The X-ray scintillator 4 is usually a GOS screen. The electronics board 5 includes function of detector pixel read-out and data transmission to PC. Because of reasonable flexibility of both detector material and GOS screen material, the whole detector 3 can be bent and configured as curved shape with pre-determined radius. But electronics board 5 on the side of the detector 3 has to be straight due to the fact that the board is rigid.

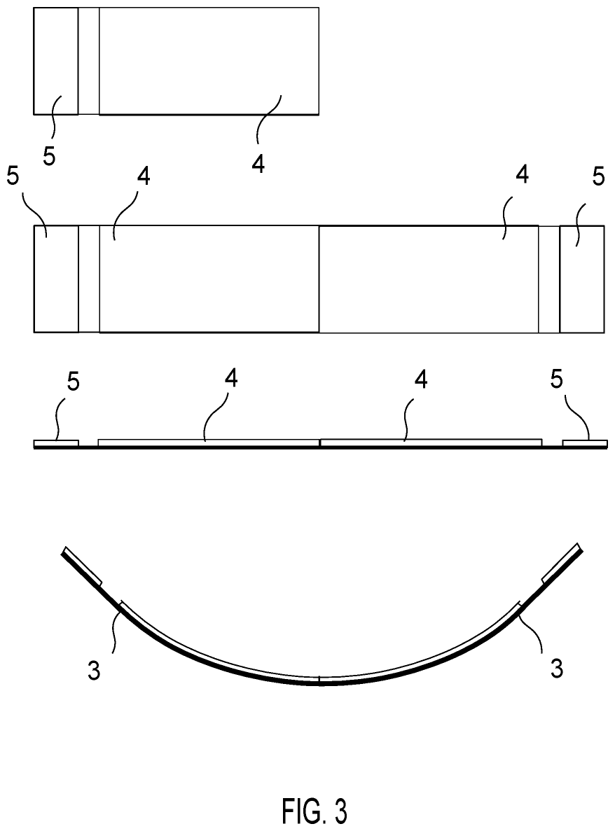

FIG. 3 shows a configuration of two sets of one-piece curved X-ray detectors 3 that are positioned head-to-head. Sometimes one set of one-piece curved X-ray detector 3 is not enough due to the length limitation. Two sets of one-piece curved detectors 3 can be positioned head-to-head and work parallel. And they can cover a larger object 8.

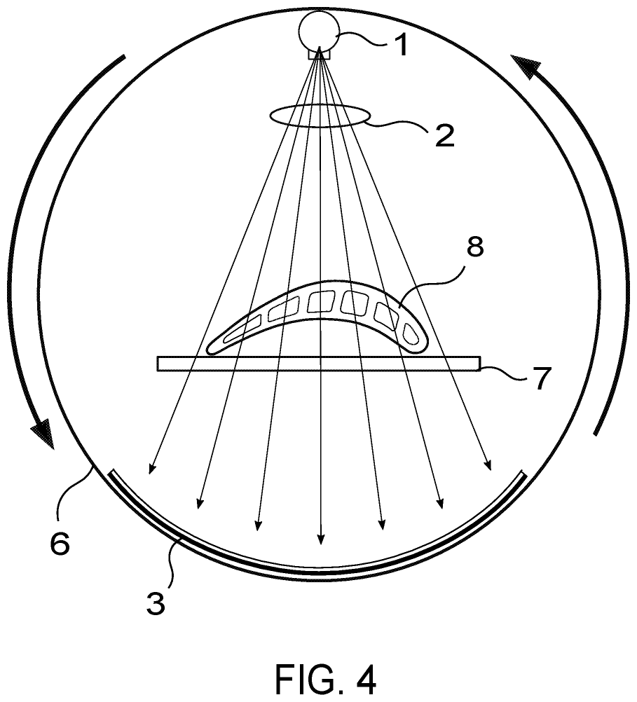

FIG. 4 shows cross section of a CT imaging apparatus with one X-ray source 1 and one-piece curved X-ray detector 3. Modern fast computers 9 can easily take multiple data acquisition interfaces so that this kind of CT imaging apparatus can also be easily constructed and connected. X-ray beam 2 covers the object 8 and the one-piece curved detector 3.

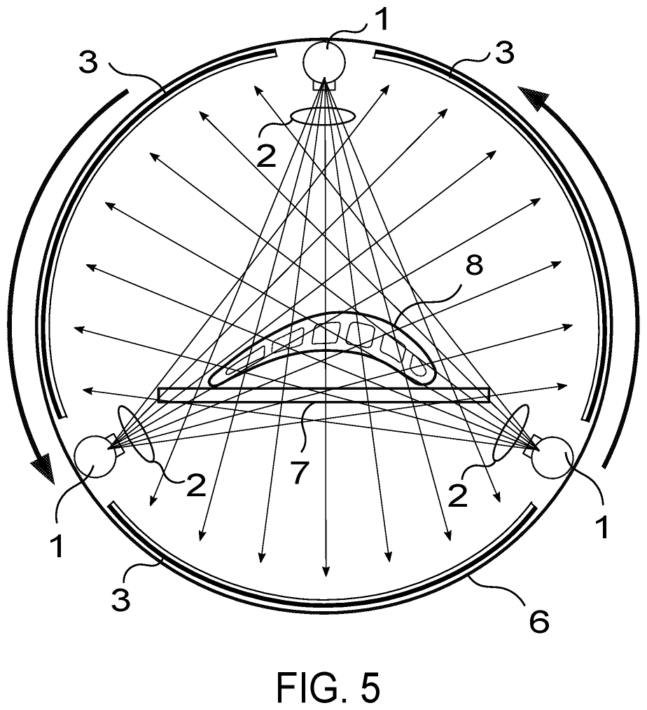

FIG. 5 shows a cross section of a CT imaging apparatus with multiple X-ray sources 1 and multiple one-piece curved detectors 3. Multiple X-ray sources 1 and multiple one-piece X-ray detectors 3 mounted on the gantry 6 can work parallel while they are rotating together.

These and other objects and advantages of the present invention will become apparent to those skilled in the art in view of the description of the best presently known mode of carrying out the invention as described herein and as illustrated in the drawings.

The present invention has been described in terms of the preferred embodiment, and it is recognized that equivalents, alternatives, and modifications, aside from those expressly stated, are possible and within the scope of the appending claims.

What is claimed is:

1. An X-ray CT imaging apparatus with one-piece curved detector comprising:
a X-ray source that can produce X-ray radiation;
one or a plurality of one-piece curved X-ray detectors;
a rotational gantry where both said X-ray source and said one-piece curved X-ray detector are mounted on;
a translational stage on which an object to be examined is placed;
one or a plurality of computer equipped with data acquisition hardware and software system;
a means for performing correction processes to X-ray helical projection data; and
a means for performing tomography image reconstruction from corrected X-ray helical projection data.

2. The X-ray CT imaging apparatus with one-piece curved detector of claim 1, wherein said one-piece curved X-ray detector includes a plurality of detector pixel elements arranged in both longitudinal axial and tangential directions of said rotational gantry.

3. The X-ray CT imaging apparatus with one-piece curved detector of claim 1, wherein said translational stage moves along longitude axial direction of said rotational gantry.

4. An X-ray CT imaging apparatus with one-piece curved detector comprising:
a plurality of X-ray sources that can produce X-ray radiation;
a plurality of one-piece curved X-ray detectors;
a rotational gantry where both said X-ray source and said one-piece curved X-ray detector are mounted on;
a translational stage on which an object to be examined is placed;
one or a plurality of computers equipped with data acquisition hardware and software system;
a means for performing correction processes to X-ray helical projection data; and
a means for performing tomography image reconstruction from corrected X-ray helical projection data.

5. A method of X-ray CT imaging apparatus with one-piece curved detector, the method comprising:
positioning an X-ray source on one side of rotational gantry;
positioning a one-piece curved detector on another side of rotational gantry;
positioning an object on a translational stage;
rotating said rotational gantry using a motion control system;
moving said translational stage along longitudinal axial direction of said gantry using a motion control system;
acquiring X-ray helical projection data of said object using a computer while said rotational gantry is rotating, said translational stage is in motion, said X-ray source is emitting radiation and said one-piece curved detector is active;
performing correction processes to X-ray helical projection data; and
performing tomography image reconstruction from corrected X-ray helical projection data.

6. The method as in claim 5, wherein the computer is equipped with imaging data acquisition hardware and software system.

7. The method as in claim 5, wherein image data acquisition system can be performed using either wired or wireless connection.
